(19) United States
(12) Patent Application Publication
(10) Pub. No.: US 2007/0173942 A1
(43) Pub. Date: Jul. 26, 2007

(54) Intervertebral prosthetic disc

(75) Inventors: Eric S. Heinz, Memphis, TN (US); Jeffrey P. Rouleau, Maple Grove, MN (US); Shannon M. Vittur, Memphis, TN (US)

Correspondence Address: LARSON NEWMAN ABEL POLANSKY & WHITE, LLP, 5914 WEST COURTYARD DRIVE, SUITE 200, AUSTIN, TX, 78730, US

(73) Assignee: SDGI HOLDINGS, INC., Wilmington, DE (US)

(21) Appl. No.: 11/340,426

(22) Filed: Jan. 26, 2006

(51) Int. Cl. A61F 2/44

(52) U.S. Cl. 623/17.15; 623/17.16

(57) ABSTRACT

An intervertebral prosthetic disc is disclosed and can be installed within an intervertebral space between an inferior vertebra and a superior vertebra. The intervertebral prosthetic disc includes a superior component that can be configured to engage the superior vertebra and an inferior component that can be configured to engage the inferior vertebra. A nucleus can be disposed between the superior component and the inferior component. The nucleus can be configured to allow relative motion between the superior component and the inferior component. Further, the intervertebral prosthetic disc can include at least one nucleus containment feature that can be configured to prevent the nucleus from migrating with respect to the superior component and the inferior component without interfering with the relative motion between the superior component and the inferior component in any direction.

### FIELD OF THE DISCLOSURE

The present disclosure relates generally to orthopedics and spinal surgery. More specifically, the present disclosure relates to intervertebral prosthetic discs.

### BACKGROUND

In human anatomy, the spine is a generally flexible column that can take tensile and compressive loads. The spine also allows bending motion and provides a place of attachment for ribs, muscles and ligaments. Generally, the spine is divided into three sections: the cervical spine, the thoracic spine and the lumbar spine. The sections of the spine are made up of individual bones called vertebrae. Also, the vertebrae are separated by intervertebral discs, which are situated between adjacent vertebrae.

The intervertebral discs function as shock absorbers and as joints. Further, the intervertebral discs can absorb the compressive and tensile loads to which the spinal column may be subjected. At the same time, the intervertebral discs can allow adjacent vertebral bodies to move relative to each other a limited amount, particularly during bending, or flexure, of the spine. Thus, the intervertebral discs are under constant muscular and/or gravitational pressure and generally, the intervertebral discs are the first parts of the lumbar spine to show signs of deterioration.

Facet joint degeneration is also common because the facet joints are in almost constant motion with the spine. In fact, facet joint degeneration and disc degeneration frequently occur together. Generally, although one may be the primary problem while the other is a secondary problem resulting from the altered mechanics of the spine, by the time surgical options are considered, both facet joint degeneration and disc degeneration typically have occurred. For example, the altered mechanics of the facet joints and/or intervertebral disc may cause spinal stenosis, degenerative spondylolisthesis, and degenerative scoliosis.

One surgical procedure for treating these conditions is spinal arthrodesis, i.e., spine fusion, which can be performed anteriorally, posteriorally, and/or laterally. The posterior procedures include in-situ fusion, posterior lateral instrumented fusion, transforaminal lumbar interbody fusion (“TLIF”) and posterior lumbar interbody fusion (“PLIF”). Solidly fusing a spinal segment to eliminate any motion at that level may alleviate the immediate symptoms, but for some patients maintaining motion may be beneficial. It is also known to surgically replace a degenerative disc or facet joint with an artificial disc or an artificial facet joint, respectively.

### BRIEF DESCRIPTION OF THE DRAWINGS

FIG. 1 is a lateral view of a portion of a vertebral column;

FIG. 2 is a lateral view of a pair of adjacent vertrebrae;

FIG. 3 is a top plan view of a vertebra;

FIG. 4 is a lateral view of a first embodiment of an intervertebral prosthetic disc;

FIG. 5 is another lateral view of the first embodiment of the intervertebral prosthetic disc;

FIG. 6 is an exploded lateral view of the first embodiment of the intervertebral prosthetic disc;

FIG. 7 is a anterior view of the first embodiment of the intervertebral prosthetic disc;

FIG. 8 is a perspective view of a superior component of the first embodiment of the intervertebral prosthetic disc;

FIG. 9 is a perspective view of an inferior component of the first embodiment of the intervertebral prosthetic disc;

FIG. 10 is an exploded lateral view of the first embodiment of the intervertebral prosthetic disc installed within an intervertebral space between a pair of adjacent vertrebrae;

FIG. 11 is a lateral view of a second embodiment of an intervertebral prosthetic disc;

FIG. 12 is another lateral view of the second embodiment of an intervertebral prosthetic disc;

FIG. 13 is an exploded lateral view of the second embodiment of the intervertebral prosthetic disc;

FIG. 14 is a anterior view of the second embodiment of the intervertebral prosthetic disc;

FIG. 15 is a perspective view of a superior component of the second embodiment of the intervertebral prosthetic disc;

FIG. 16 is a perspective view of an inferior component of the second embodiment of the intervertebral prosthetic disc;

FIG. 17 is a lateral view of a third embodiment of an intervertebral prosthetic disc;

FIG. 18 is another lateral view of the third embodiment of an intervertebral prosthetic disc;

FIG. 19 is an exploded lateral view of the third embodiment of the intervertebral prosthetic disc;

FIG. 20 is a anterior view of the third embodiment of the intervertebral prosthetic disc;

FIG. 21 is a perspective view of a superior component of the third embodiment of the intervertebral prosthetic disc; and

FIG. 22 is a perspective view of an inferior component of the third embodiment of the intervertebral prosthetic disc.

### DETAILED DESCRIPTION OF THE DRAWINGS

An intervertebral prosthetic disc is disclosed and can be installed within an intervertebral space between an inferior vertebra and a superior vertebra. The intervertebral prosthetic disc includes a superior component that can be configured to engage the superior vertebra and an inferior component that can be configured to engage the inferior vertebra. A nucleus can be disposed between the superior component and the inferior component. The nucleus can be configured to allow relative motion between the superior component and the inferior component. Further, the intervertebral prosthetic disc can include at least one nucleus containment feature that can be configured to prevent the nucleus from migrating with respect to the superior component and the inferior component without interfering with the relative motion between the superior component and the inferior component in any direction.

In another embodiment, an intervertebral prosthetic disc is disclosed and can be installed within an intervertebral space between an inferior vertebra and a superior vertebra. In this embodiment, the intervertebral prosthetic disc includes a superior component that can be configured to engage the superior vertebra. Further, the superior component can include a superior depression established therein and the superior depression can include an anterior rim and a posterior rim. Also, the intervertebral prosthetic disc can include an inferior component that can be configured to engage the inferior vertebra. The inferior component can include an inferior depression established therein and the inferior depression can include an anterior rim and a posterior rim. In this particular embodiment, a nucleus can be disposed between the superior component and the inferior component. The nucleus can be configured to engage the superior depression and the inferior depression and the nucleus can be configured to allow relative motion between the superior component and the inferior component. Further, at least one superior nucleus containment post can extend from the superior component and the superior nucleus containment post can be configured to prevent the nucleus from migrating with respect to the superior component and the inferior component.

In yet another embodiment, an intervertebral prosthetic disc is disclosed and can be installed within an intervertebral space between adjacent first and second vertebrae. The intervertebral prosthetic disc can include a first component that can be configured to engage the first vertebra. The first component can include a depression established therein and the depression can include an anterior rim and a posterior rim. Further, the intervertebral prosthetic disc can include a second component that can be configured to engage the second vertebra. The second component can include a depression established therein and the depression can include an anterior rim and a posterior rim. Also, a nucleus can be disposed between the first component and the second component. The nucleus can be configured to engage the depression of the first component and the depression of the second component and the nucleus can be configured to allow relative motion between the first component and the second component. Moreover, a first nucleus containment rail can extend from at least one of the first component or the second component. The first nucleus containment rail can be configured to prevent the nucleus from migrating with respect to the first component and the second component.

In still another embodiment, an intervertebral prosthetic disc is disclosed and can be installed within an intervertebral space between adjacent first and second vertebrae. The intervertebral prosthetic disc can include a superior component that can be configured to engage the superior vertebra. Also, the superior component can include a superior projection that extends therefrom. Moreover, the intervertebral prosthetic disc can include an inferior component that is configured to engage the inferior vertebra. The inferior component can include an inferior projection that extends therefrom. Further, a nucleus can be disposed between the superior component and the inferior component. The nucleus can be configured to engage the superior projection and the inferior projection and wherein the nucleus can be configured to allow relative motion between the superior component and the inferior component. Additionally, an inferior nucleus containment rail can extend from the inferior component. The inferior nucleus containment rail can be configured to prevent the nucleus from migrating with respect to the superior component and the inferior component.

Description of Relevant Anatomy

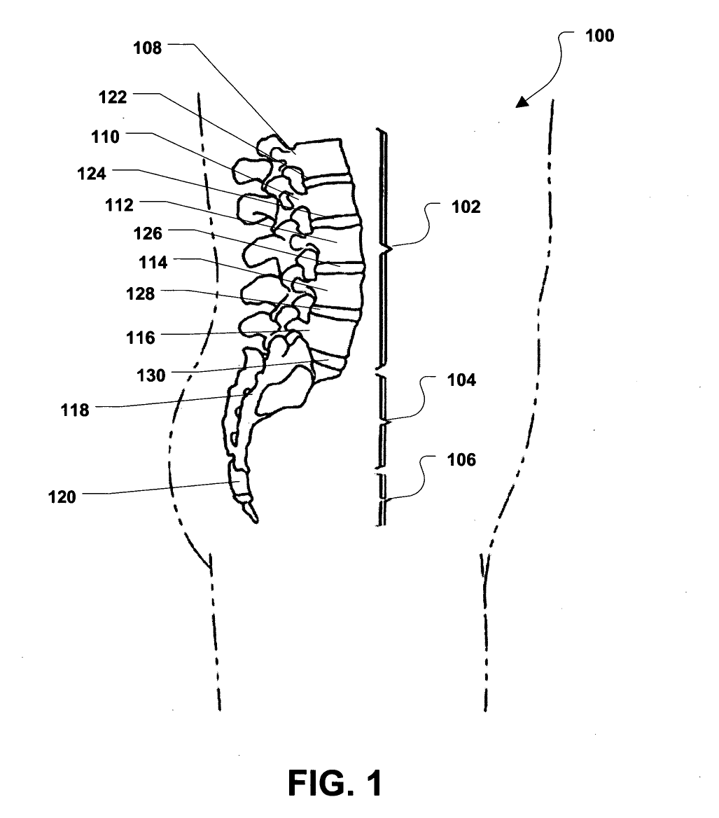

Referring initially to FIG. 1, a portion of a vertebral column, designated 100, is shown. As depicted, the vertebral column 100 includes a lumber region 102, a sacral region 104, and a coccygeal region 106. As is known in the art, the vertebral column 100 also includes a cervical region and a thoracic region. For clarity and ease of discussion, the cervical region and the thoracic region are not illustrated.

As shown in FIG. 1, the lumbar region 102 includes a first lumber vertebra 108, a second lumbar vertebra 110, a third lumbar vertebra 112, a fourth lumbar vertebra 114, and a fifth lumbar vertebra 116. The sacral region 104 includes a sacrum 118. Further, the coccygeal region 106 includes a coccyx 120.

As depicted in FIG. 1, a first intervertebral lumbar disc 122 is disposed between the first lumber vertebra 108 and the second lumbar vertebra 110. A second intervertebral lumbar disc 124 is disposed between the second lumbar vertebra 110 and the third lumbar vertebra 112. A third intervertebral lumbar disc 126 is disposed between the third lumbar vertebra 112 and the fourth lumbar vertebra 114. Further, a fourth intervertebral lumbar disc 128 is disposed between the fourth lumbar vertebra 114 and the fifth lumbar vertebra 116. Additionally, a fifth intervertebral lumbar disc 130 is disposed between the fifth lumbar vertebra 116 and the sacrum 118.

In a particular embodiment, if one of the intervertebral lumbar discs 122, 124, 126, 128, 130 is diseased, degenerated, damaged, or otherwise in need of replacement, that intervertebral lumbar disc 122, 124, 126, 128, 130 can be at least partially removed and replaced with an intervertebral prosthetic disc according to one or more of the embodiments described herein. In a particular embodiment, a portion of the intervertebral lumbar disc 122, 124, 126, 128, 130 can be removed via a discectomy, or a similar surgical procedure, well known in the art. Further, removal of intervertebral lumbar disc material can result in the formation of an intervertebral space (not shown) between two adjacent lumbar vertebrae.

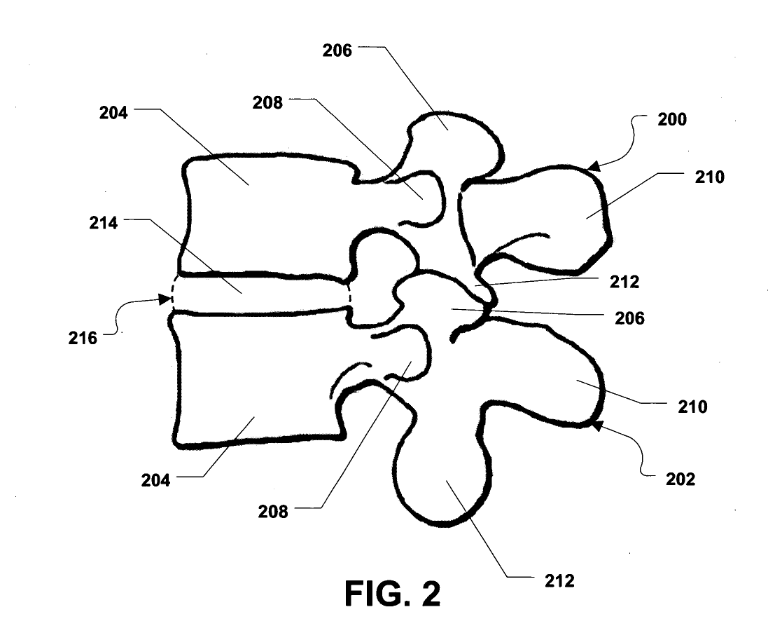

FIG. 2 depicts a detailed lateral view of two adjacent vertebrae, e.g., two of the lumbar vertebra 108, 110, 112, 114, 116 shown in FIG. 1. FIG. 2 illustrates a superior vertebra 200 and an inferior vertebra 202. As shown, each vertebra 200, 202 includes a vertebral body 204, a superior articular process 206, a transverse process 208, a spinous process 210 and an inferior articular process 212. FIG. 2 further depicts an intervertebral space 214 that can be established between the superior vertebra 200 and the inferior vertebra 202 by removing an intervertebral disc 216 (shown in dashed lines). As described in greater detail below, an intervertebral prosthetic disc according to one or more of the embodiments described herein can be installed within the intervertebral space 212 between the superior vertebra 200 and the inferior vertebra 202.

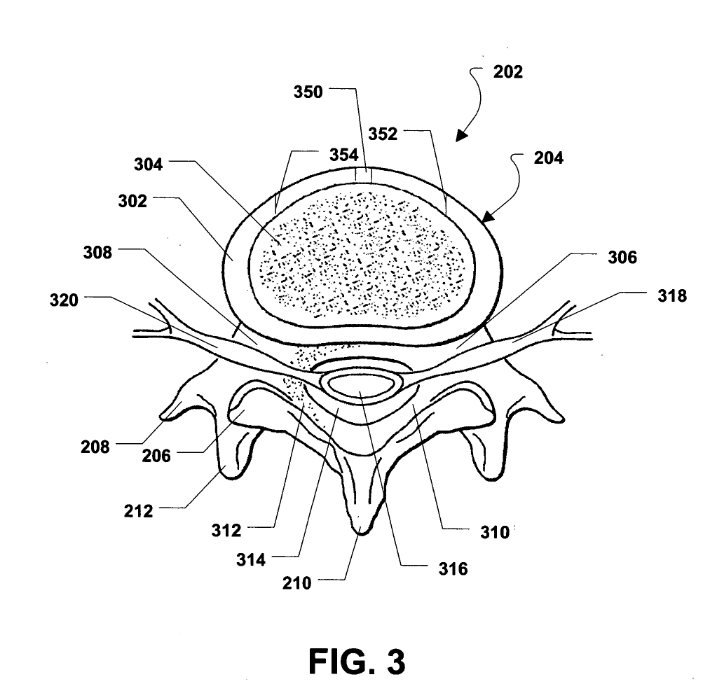

Referring to FIG. 3, a vertebra, e.g., the inferior vertebra 202 (FIG. 2), is illustrated. As shown, the vertebral body 204 of the inferior vertebra 202 includes a cortical rim 302 composed of cortical bone. Also, the vertebral body 204 includes cancellous bone 304 within the cortical rim 302. The cortical rim 302 is often referred to as the apophyseal rim or apophyseal ring. Further, the cancellous bone 304 is softer than the cortical bone of the cortical rim 302.

As illustrated in FIG. 3, the inferior vertebra 202 further includes a first pedicle 306, a second pedicle 308, a first lamina 310, and a second lamina 312. Further, a vertebral foramen 314 is established within the inferior vertebra 202. A spinal cord 316 passes through the vertebral foramen 314. Moreover, a first nerve root 318 and a second nerve root 320 extend from the spinal cord 316.

It is well known in the art that the vertebrae that make up the vertebral column have slightly different appearances as they range from the cervical region to the lumbar region of the vertebral column. However, all of the vertebrae, except the first and second cervical vertebrae, have the same basic structures, e.g., those structures described above in conjunction with FIG. 2 and FIG. 3. The first and second cervical vertebrae are structurally different than the rest of the vertebrae in order to support a skull.

FIG. 3 further depicts a keel groove 350 that can be established within the cortical rim 302 of the inferior vertebra 202. Further, a first corner cut 352 and a second corner cut 354 can be established within the cortical rim 302 of the inferior vertebra 202. In a particular embodiment, the keel groove 350 and the corner cuts 352, 354 can be established during surgery to install an intervertebral prosthetic disc according to one or more of the embodiments described herein. The keel groove 350 can be established using a keel cutting device, e.g., a keel chisel designed to cut a groove in a vertebra, prior to the installation of the intervertebral prosthetic disc. Further, the keel groove 350 is sized and shaped to receive and engage a keel, described in detail below, that extends from an intervertebral prosthetic disc according to one or more of the embodiments described herein. The keel groove 350 can cooperate with a keel to facilitate proper alignment of an intervertebral prosthetic disc within an intervertebral space between an inferior vertebra and a superior vertebra.

### Description of a First Embodiment

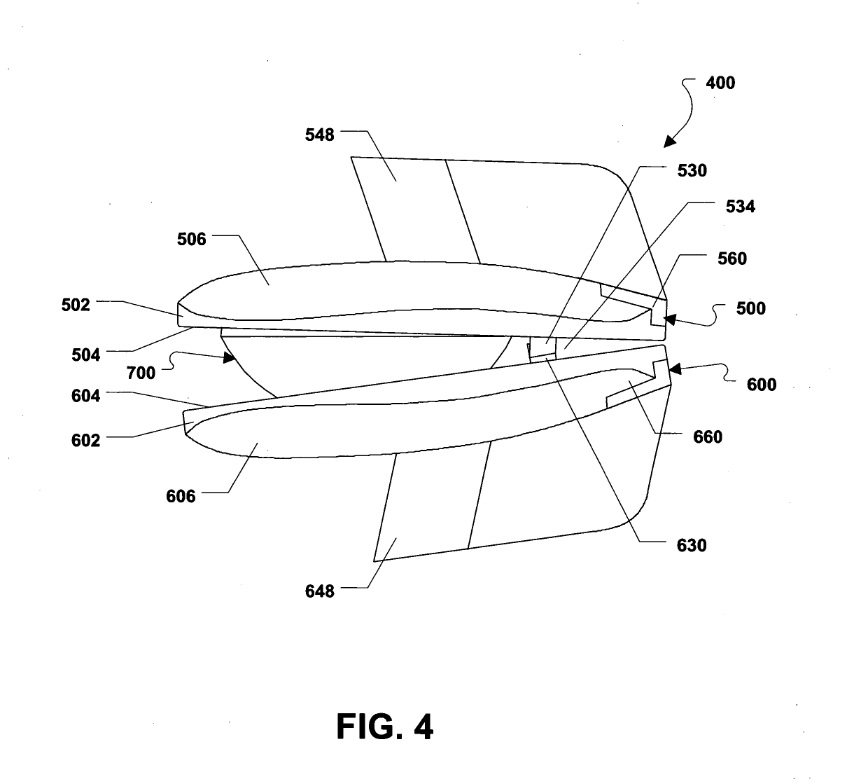

Referring to FIGS. 4 through 9 a first embodiment of an intervertebral prosthetic disc is shown and is generally designated 400. As illustrated, the intervertebral prosthetic disc 400 includes a superior component 500, an inferior component 600, and a nucleus 700 disposed, or otherwise installed, there between. In a particular embodiment, the articular halves 500, 600 and the nucleus 700 can be made from one or more extended use approved medical materials. For example, the materials can be metal containing materials, polymer materials, or composite materials that include metals, polymers, or combinations of metals and polymers.

In a particular embodiment, the metal containing materials can be metals. Further, the metal containing materials can be ceramics. Also, the metals can be pure metals or metal alloys. The pure metals can include titanium. Moreover, the metal alloys can include stainless steel, a cobalt-chrome-molybdenum alloy, e.g., ASTM F-999 or ASTM F-75, a titanium alloy, or a combination thereof.

The polymer materials can include polyurethane materials, polyolefin materials, polyether materials, silicone materials, or combinations thereof. Further, the polyolefin materials can include polypropylene, polyethylene, halogenated polyolefin, flouropolyolefin, or a combination thereof. The polyether materials can include polyetherketone (PEK), polyetheretherketone (PEEK), polyetherketoneketone (PEKK), polyaryletherketone (PAEK), or a combination thereof. Alternatively, the articular halves 500, 600 can be made from any other substantially rigid biocompatible materials.

In a particular embodiment, the superior component 500 includes a superior support plate 502 that has a superior articular surface 504 and a superior bearing surface 506. In a particular embodiment, the superior articular surface 504 can be substantially flat and the superior bearing surface 506 can be generally curved. In an alternative embodiment, at least a portion of the superior articular surface 504 can be generally curved and the superior bearing surface 506 can be substantially flat.

In a particular embodiment, after installation, the superior bearing surface 506 can be in direct contact with vertebral bone, e.g., cortical bone and cancellous bone. Further, the superior bearing surface 506 can be coated with a bone-growth promoting substance, e.g., a hydroxyapatite coating formed of calcium phosphate. In a particular embodiment, the superior bearing surface 506 does not include proteins, e.g., bone morphogenetic protein (BMP). Additionally, the superior bearing surface 506 can be roughened prior to being coated with the bone-growth promoting substance to further enhance bone on-growth or in-growth. In a particular embodiment, the roughening process can include acid etching; knurling; application of a bead coating (porous or non-porous), e.g., cobalt chrome beads; application of a roughening spray, e.g., titanium plasma spray (TPS); laser blasting; or any other similar process or method.

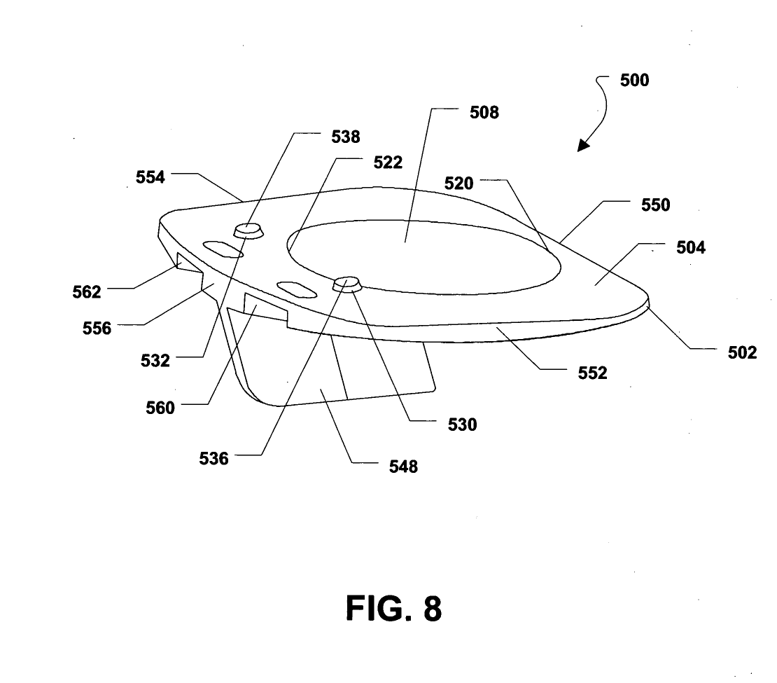

As illustrated in FIG. 8, a superior depression 508 is established within the superior articular surface 504 of the superior support plate 502. In a particular embodiment, the superior depression 508 has an arcuate shape. For example, the superior depression 508 can have a hemispherical shape, an elliptical shape, a cylindrical shape, or any combination thereof.

As further shown in FIG. 8, the superior depression 508 includes an anterior rim 520 and a poster rim 522. Further, a first superior nucleus containment post 530 and a second superior nucleus containment post 532 extend from the superior articular surface 504 adjacent to the anterior rim 520 of the depression. In a particular embodiment, each superior nucleus containment post 530, 532 extends into a gap 534 that can be established between the superior component 500 and the inferior component 600 posterior to the nucleus 700. Further, each superior nucleus containment post 530, 532 can include a slanted upper surface 536, 538. In a particular embodiment, the slanted upper surface 536, 538 of each superior nucleus containment post 530, 532 prevents each nucleus containment post 530, 532 from interfering with the motion of the inferior component 600 with respect to the superior component 500.

FIG. 4 through FIG. 8 indicate that the superior component 500 can include a superior keel 548 that extends from superior bearing surface 506. During installation, described below, the superior keel 548 can at least partially engage a keel groove that can be established within a cortical rim of a vertebra. Further, the superior keel 548 can be coated with a bone-growth promoting substance, e.g., a hydroxyapatite coating formed of calcium phosphate. In a particular embodiment, the superior keel 548 does not include proteins, e.g., bone morphogenetic protein (BMP). Additionally, the superior keel 548 can be roughened prior to being coated with the bone-growth promoting substance to further enhance bone on-growth or in-growth. In a particular embodiment, the roughening process can include acid etching; knurling; application of a bead coating (porous or non-porous), e.g., cobalt chrome beads; application of a roughening spray, e.g., titanium plasma spray (TPS); laser blasting; or any other similar process or method.

In a particular embodiment, the superior component 500, shown in FIG. 8, can be generally rectangular in shape. For example, the superior component 500 can have a substantially straight posterior side 550. A first substantially straight lateral side 552 and a second substantially straight lateral side 554 can extend substantially perpendicularly from the posterior side 550 to an anterior side 556. In a particular embodiment, the anterior side 556 can curve outward such that the superior component 500 is wider through the middle than along the lateral sides 552, 554. Further, in a particular embodiment, the lateral sides 552, 554 are substantially the same length.

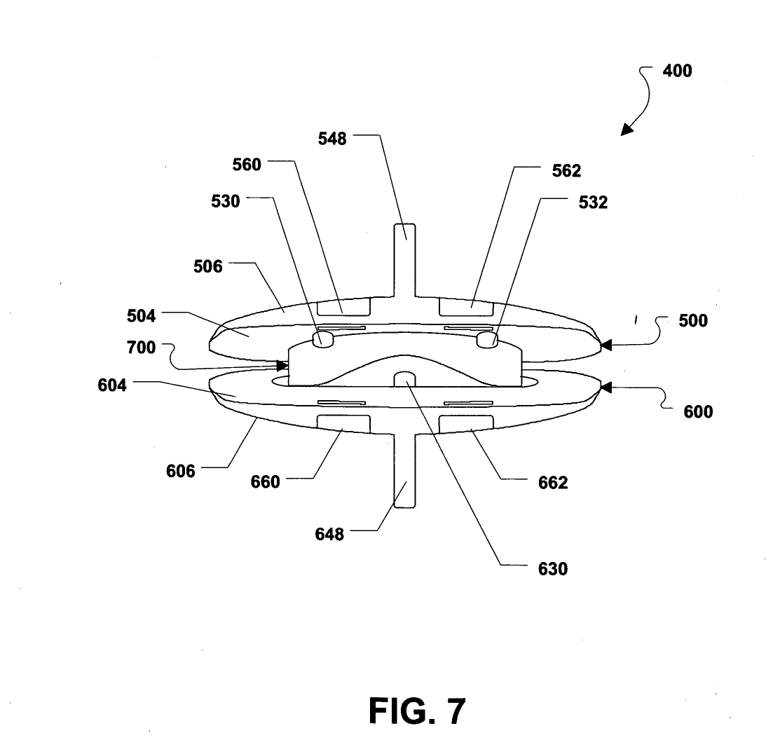

FIG. 7 shows that the superior component 500 can include a first implant inserter engagement hole 560 and a second implant inserter engagement hole 562. In a particular embodiment, the implant inserter engagement holes 560, 562 are configured to receive a correspondingly shaped arm that extends from an implant inserter (not shown) that can be used to facilitate the proper installation of an intervertebral prosthetic disc, e.g., the intervertebral prosthetic disc 400 shown in FIG. 4 through FIG. 9.

In a particular embodiment, the inferior component 600 includes an inferior support plate 602 that has an inferior articular surface 604 and an inferior bearing surface 606. In a particular embodiment, the inferior articular surface 604 can be substantially flat and the inferior bearing surface 606 can be generally curved. In an alternative embodiment, at least a portion of the inferior articular surface 604 can be generally curved and the inferior bearing surface 606 can be substantially flat.

In a particular embodiment, after installation, the inferior bearing surface 606 can be in direct contact with vertebral bone, e.g., cortical bone and cancellous bone. Further, the inferior bearing surface 606 can be coated with a bone-growth promoting substance, e.g., a hydroxyapatite coating formed of calcium phosphate. In a particular embodiment, the inferior bearing surface 606 does not include proteins, e.g., bone morphogenetic protein (BMP). Additionally, the inferior bearing surface 606 can be roughened prior to being coated with the bone-growth promoting substance to further enhance bone on-growth or in-growth. In a particular embodiment, the roughening process can include acid etching; knurling; application of a bead coating (porous or non-porous), e.g., cobalt chrome beads; application of a roughening spray, e.g., titanium plasma spray (TPS); laser blasting; or any other similar process or method.

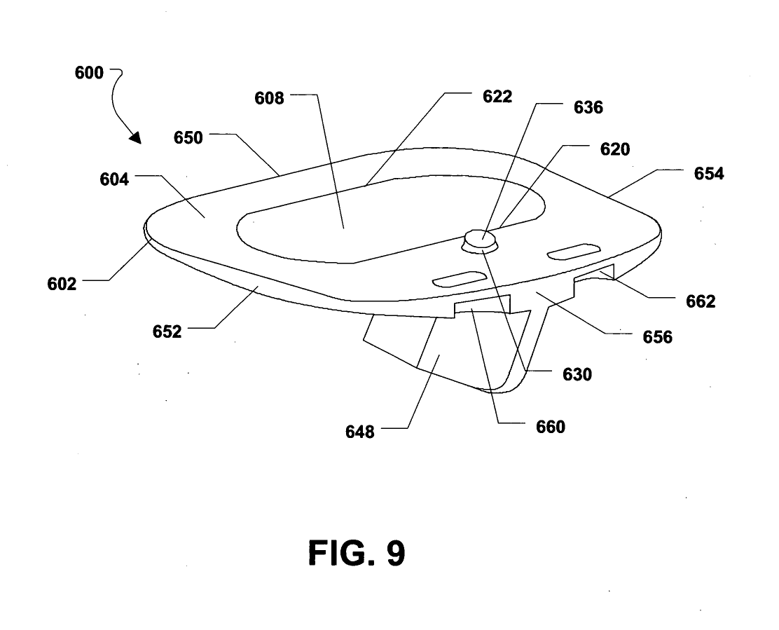

As illustrated in FIG. 9, an inferior depression 608 is established within the inferior articular surface 604 of the inferior support plate 602. In a particular embodiment, the inferior depression 608 has an arcuate shape. For example, the inferior depression 608 can have a hemispherical shape, an elliptical shape, a cylindrical shape, or any combination thereof.

As further shown in FIG. 9, the inferior depression 608 includes an anterior rim 620 and a poster rim 622. Further, an inferior nucleus containment post 630 extends from the inferior articular surface 604 adjacent to the anterior rim 620 of the inferior depression 608. In a particular embodiment, the inferior nucleus containment post 630 extends into the gap 534 between the superior component 500 and the inferior component 600 posterior to the nucleus 700. Further, the inferior nucleus containment post 630 can include a slanted upper surface 636. In a particular embodiment, the slanted upper surface 636 of the inferior nucleus containment post 630 can prevent the inferior nucleus containment post 630 from interfering with the motion of the superior component 500 with respect to the inferior component 600.

FIG. 4 through FIG. 6 and FIG. 9 indicate that the inferior component 600 can include an inferior keel 648 that extends from inferior bearing surface 606. During installation, described below, the inferior keel 648 can at least partially engage a keel groove that can be established within a cortical rim of a vertebra, e.g., the keel groove 350 shown in FIG. 3. Further, the inferior keel 548 can be coated with a bone-growth promoting substance, e.g., a hydroxyapatite coating formed of calcium phosphate. In a particular embodiment, the inferior keel 548 does not include proteins, e.g., bone morphogenetic protein (BMP). Additionally, the inferior keel 548 can be roughened prior to being coated with the bone-growth promoting substance to further enhance bone on-growth or in-growth. In a particular embodiment, the roughening process can include acid etching; knurling; application of a bead coating (porous or non-porous), e.g., cobalt chrome beads; application of a roughening spray, e.g., titanium plasma spray (TPS); laser blasting; or any other similar process or method.

In a particular embodiment, the inferior component 600, shown in FIG. 9, can be shaped to match the shape of the inferior component 500, shown in FIG. 8. Further, the inferior component 600 can be generally rectangular in shape. For example, the inferior component 600 can have a substantially straight posterior side 650. A first substantially straight lateral side 652 and a second substantially straight lateral side 654 can extend substantially perpendicularly from the posterior side 650 to an anterior side 656. In a particular embodiment, the anterior side 656 can curve outward such that the inferior component 600 is wider through the middle than along the lateral sides 652, 654. Further, in a particular embodiment, the lateral sides 652, 654 are substantially the same length.

FIG. 7 shows that the inferior component 600 can include a first implant inserter engagement hole 660 and a second implant inserter engagement hole 662. In a particular embodiment, the implant inserter engagement holes 660, 662 are configured to receive a correspondingly shaped arm that extends from an implant inserter (not shown) that can be used to facilitate the proper installation of an intervertebral prosthetic disc, e.g., the intervertebral prosthetic disc 400 shown in FIG. 4 through FIG. 9.

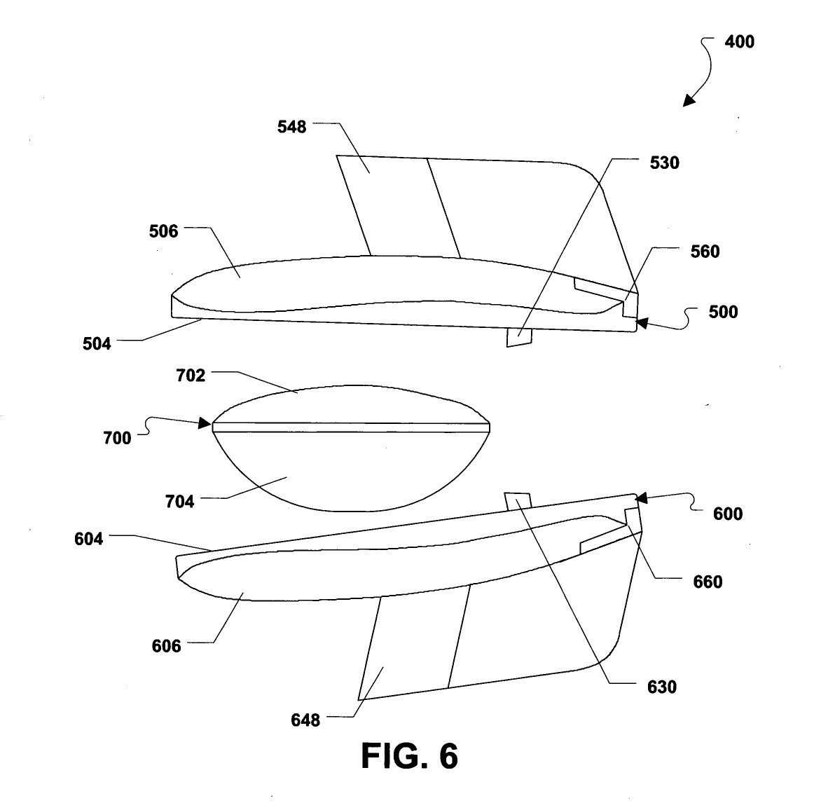

FIG. 6 shows that the nucleus 700 can include a superior bearing surface 702 and an inferior bearing surface 704. In a particular embodiment, the superior bearing surface 702 and the inferior bearing surface 704 can each have an arcuate shape. For example, the superior bearing surface 702 of the nucleus 700 and the inferior bearing surface 704 of the nucleus 700 can have a hemispherical shape, an elliptical shape, a cylindrical shape, or any combination thereof. Further, in a particular embodiment, the superior bearing surface 702 can be curved to match the superior depression 508 of the superior component 500. Also, in a particular embodiment, the inferior bearing surface 704 of the nucleus can be curved to match the inferior depression 608 of the inferior component 600.

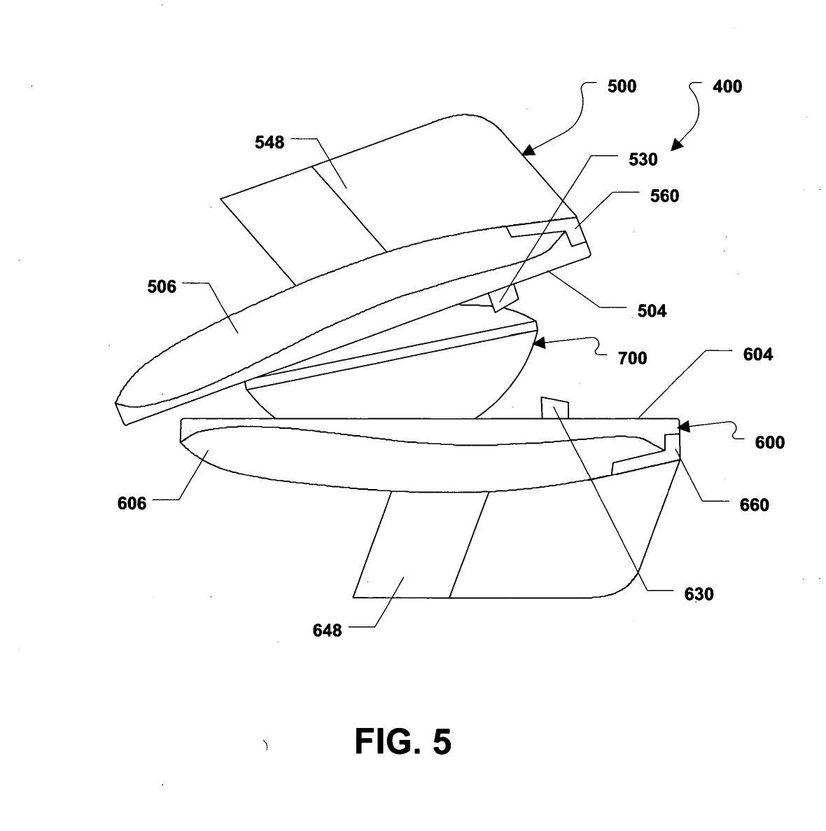

As shown in FIG. 4, the superior bearing surface 702 of the nucleus 700 can engage the superior depression 508 and allow the superior component 500 to move relative to the nucleus 700. Also, the inferior bearing surface 704 of the nucleus 700 can engage the inferior depression 608 and allow the inferior component 600 to move relative to the nucleus 700. Accordingly, the nucleus 700 can engage the superior component 500 and the inferior component 600 and the nucleus 700 can allow the superior component 500 to rotate with respect to the inferior component 600, as shown in FIG. 5.

In a particular embodiment, the superior nucleus containment posts 530, 532 on the superior component 500 and the inferior nucleus containment post 630 on the inferior component 600 can prevent the nucleus 700 from migrating, or moving, with respect to the superior component 500 and the inferior component 600. In other words, the superior nucleus containment posts 530, 532 and the inferior nucleus containment post 630 can prevent the nucleus 700 from moving out of the superior depression 508, the inferior depression 608, or a combination thereof.

Further, the superior nucleus containment posts 530, 532 and the inferior nucleus containment post 630 can prevent the nucleus 700 from being expelled from the intervertebral prosthetic device 400. In other words, the superior nucleus containment posts 530, 532 and the inferior nucleus containment post 630 can prevent the nucleus 700 from being completely ejected from the intervertebral prosthetic device 400 while the superior component 500 and the inferior component 600 move with respect to each other.

Although only three total nucleus containment posts are shown, either or both of the superior component 500 and the inferior component 600 can include one or more additional containment posts disposed at various positions around the perimeter of the superior depression 508 and/or the inferior depression 608 to prevent expulsion of the nucleus 700 in any one of multiple directions.

In a particular embodiment, the overall height of the intervertebral prosthetic device 400 can be in a range from fourteen millimeters to forty-six millimeters (14-46 mm). Further, the installed height of the intervertebral prosthetic device 400 can be in a range from eight millimeters to sixteen millimeters (8-16 mm). In a particular embodiment, the installed height can be substantially equivalent to the distance between an inferior vertebra and a superior vertebra when the intervertebral prosthetic device 400 is installed there between.

In a particular embodiment, the length of the intervertebral prosthetic device 400, e.g., along a longitudinal axis, can be in a range from thirty millimeters to forty millimeters (30-40 mm). Additionally, the width of the intervertebral prosthetic device 400, e.g., along a lateral axis, can be in a range from twenty-five millimeters to forty millimeters (25-40 mm). Moreover, in a particular embodiment, each keel 548, 648 can have a height in a range from three millimeters to fifteen millimeters (3-15 mm).

Installation of the First Embodiment within an Intervertebral Space

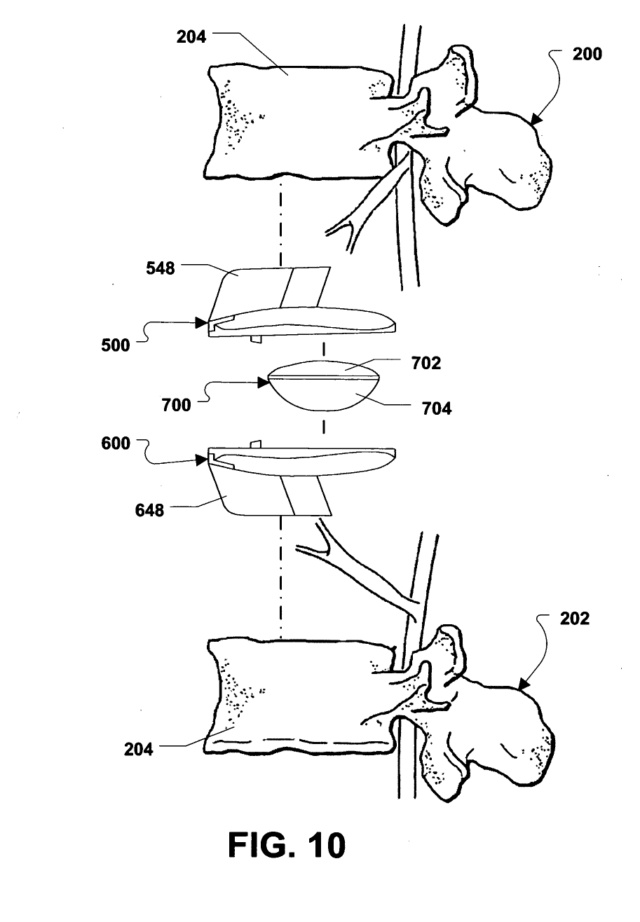

Referring to FIG. 10, an intervertebral prosthetic disc is shown between the superior vertebra 200 and the inferior vertebra 202, previously introduced and described in conjunction with FIG. 2. In a particular embodiment, the intervertebral prosthetic disc is the intervertebral prosthetic disc 400 described in conjunction with FIG. 4 through FIG. 9. Alternatively, the intervertebral prosthetic disc can be an intervertebral prosthetic disc according to any of the embodiments disclosed herein.

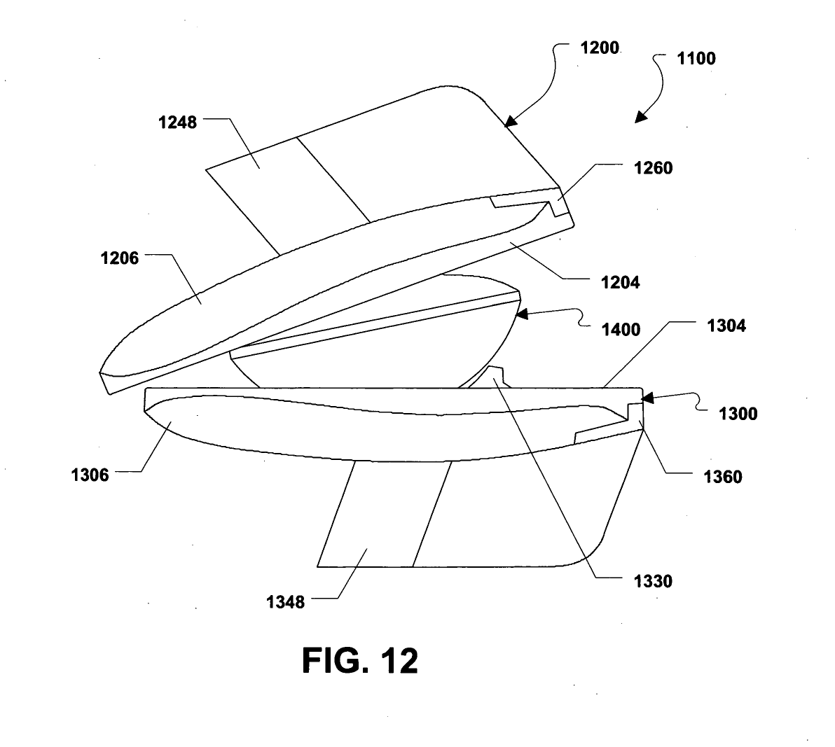

As shown in FIG. 10 through FIG. 12, the intervertebral prosthetic disc 400 is installed within the intervertebral space 214 that can be established between the superior vertebra 200 and the inferior vertebra 202 by removing vertebral disc material (not shown). FIG. 10 shows that the superior keel 548 of the superior component 500 can at least partially engage the cancellous bone and cortical rim of the superior vertebra 200. Further, in a particular embodiment, the superior keel 548 of the superior component 500 can at least partially engage a superior keel groove that can be established within the vertebral body 204 of the superior vertebra 202. In a particular embodiment, the vertebral body 204 can be further cut to allow the superior support plate 502 of the superior component 500 to be at least partially recessed into the vertebral body 204 of the superior vertebra 200.

Also, in a particular embodiment, the inferior keel 648 of the inferior component 600 can at least partially engage the cancellous bone and cortical rim of the inferior vertebra 202. Further, in a particular embodiment, the inferior keel 648 of the inferior component 600 can at least partially engage an inferior keel groove that can be established within the vertebral body 204 of the inferior vertebra 202. In a particular embodiment, the vertebral body 204 can be further cut to allow the inferior support plate 602 of the inferior component 600 to be at least partially recessed into the vertebral body 204 of the inferior vertebra 200.

As illustrated in FIG. 10, the nucleus 700 of the intervertebral prosthetic disc 400 can at least partially engage the superior depression 508 of the superior component 500 and the inferior depression 608 of the inferior component 600. It is to be appreciated that when the intervertebral prosthetic disc 400 is installed between the superior vertebra 200 and the inferior vertebra 202, the intervertebral prosthetic disc 400 allows relative motion between the superior vertebra 200 and the inferior vertebra 202. Specifically, the configuration of the superior component 500 and the inferior component 600 allows the superior component 500 to rotate with respect to the inferior component 600. As such, the superior vertebra 200 can rotate with respect to the inferior vertebra 202.

In a particular embodiment, the intervertebral prosthetic disc 400 can allow angular movement in any radial direction relative to the intervertebral prosthetic disc 400. Further, as depicted in FIG. 10 through 12, the inferior component 600 can be placed on the inferior vertebra 202 so that the center of rotation of the inferior component 600 is substantially aligned with the center of rotation of the inferior vertebra 202. Similarly, the superior component 500 can be placed relative to the superior vertebra 200 so that the center of rotation of the superior component 500 is substantially aligned with the center of rotation of the superior vertebra 200. Accordingly, when the vertebral disc, between the inferior vertebra 202 and the superior vertebra 200, is removed and replaced with the intervertebral prosthetic disc 400 the relative motion of the vertebrae 200, 202 provided by the vertebral disc is substantially replicated.

During the relative motion of the superior component 500 and the inferior component 600, the superior nucleus containment posts 530, 532 on the superior component 500 and the inferior nucleus containment post 630 on the inferior component 600 can prevent nucleus 700 migration, nucleus 700 expulsion, or any other unwanted movement of the nucleus 700 with respect to the superior component 500 and the inferior component 600.

### Description of a Second Embodiment

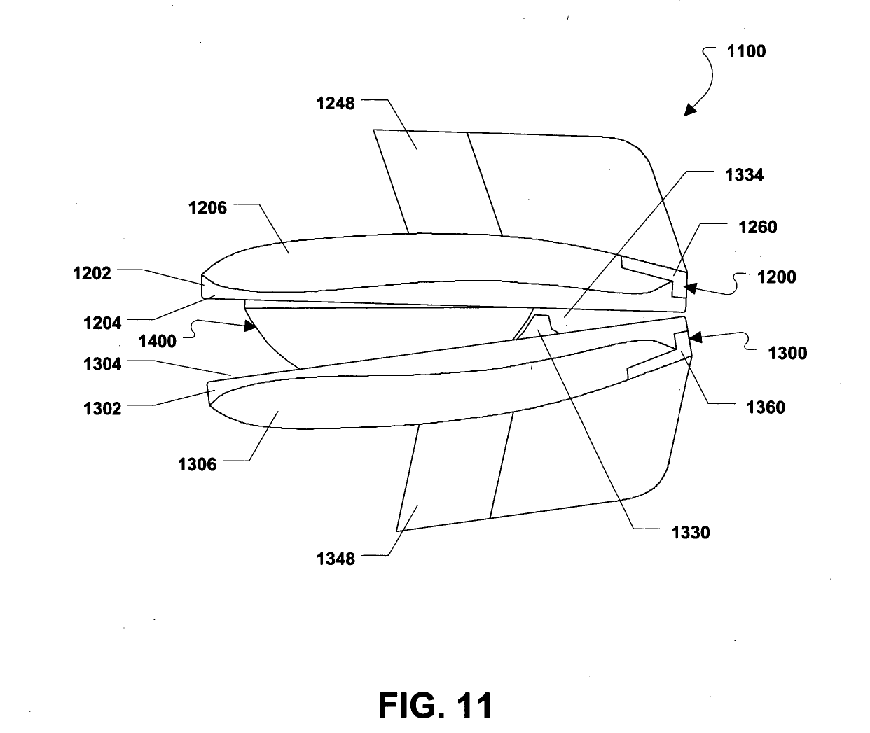

Referring to FIGS. 11 through 16 a second embodiment of an intervertebral prosthetic disc is shown and is generally designated 1100. As illustrated, the intervertebral prosthetic disc 1100 includes a superior component 1200, an inferior component 1300, and a nucleus 1400 disposed, or otherwise installed, there between. In a particular embodiment, the articular halves 1200, 1300 and the nucleus 1400 can be made from one or more extended use approved medical materials. For example, the materials can be metal containing materials, polymer materials, or composite materials that include metals, polymers, or combinations of metals and polymers.

In a particular embodiment, the metal containing materials can be metals. Further, the metal containing materials can be ceramics. Also, the metals can be pure metals or metal alloys. The pure metals can include titanium. Moreover, the metal alloys can include stainless steel, a cobalt-chrome-molybdenum alloy, e.g., ASTM F-999 or ASTM F-75, a titanium alloy, or a combination thereof.

The polymer materials can include polyurethane materials, polyolefin materials, polyether materials, silicone materials, or combinations thereof. Further, the polyolefin materials can include polypropylene, polyethylene, halogenated polyolefin, flouropolyolefin, or a combination thereof. The polyether materials can include polyetherketone (PEK), polyetheretherketone (PEEK), polyetherketoneketone (PEKK), polyaryletherketone (PAEK), or a combination thereof. Alternatively, the articular halves 1200, 1300 can be made from any other substantially rigid biocompatible materials.

In a particular embodiment, the superior component 1200 includes a superior support plate 1202 that has a superior articular surface 1204 and a superior bearing surface 1206. In a particular embodiment, the superior articular surface 1204 can be substantially flat and the superior bearing surface 1206 can be generally curved. In an alternative embodiment, at least a portion of the superior articular surface 1204 can be generally curved and the superior bearing surface 1206 can be substantially flat.

In a particular embodiment, after installation, the superior bearing surface 1206 can be in direct contact with vertebral bone, e.g., cortical bone and cancellous bone. Further, the superior bearing surface 1206 can be coated with a bone-growth promoting substance, e.g., a hydroxyapatite coating formed of calcium phosphate. In a particular embodiment, the superior bearing surface 1206 does not include proteins, e.g., bone morphogenetic protein (BMP). Additionally, the superior bearing surface 1206 can be roughened prior to being coated with the bone-growth promoting substance to further enhance bone on-growth or in-growth. In a particular embodiment, the roughening process can include acid etching; knurling; application of a bead coating (porous or non-porous), e.g., cobalt chrome beads; application of a roughening spray, e.g., titanium plasma spray (TPS); laser blasting; or any other similar process or method.

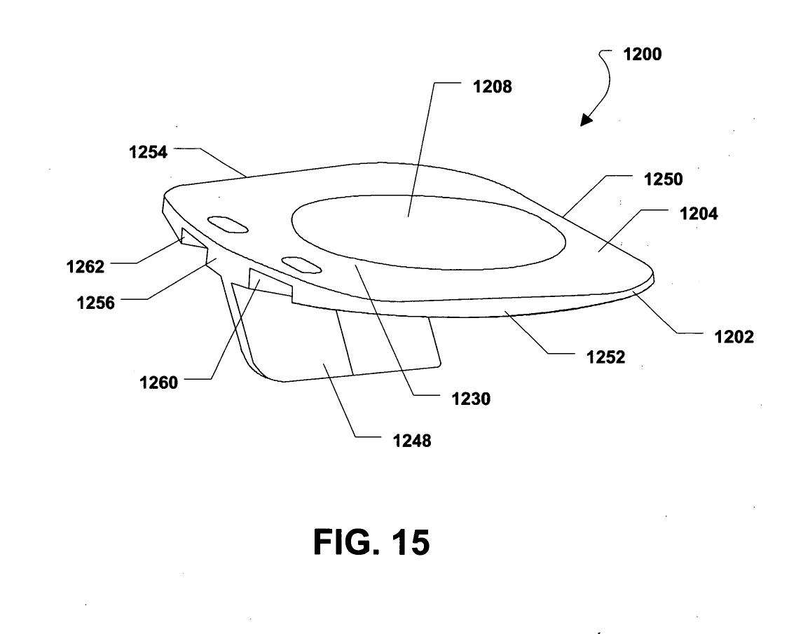

As illustrated in FIG. 15, a superior depression 1208 is established within the superior articular surface 1204 of the superior support plate 1202. In a particular embodiment, the superior depression 1208 has an arcuate shape. For example, the superior depression 1208 can have a hemispherical shape, an elliptical shape, a cylindrical shape, or any combination thereof.

FIG. 11 through FIG. 15 indicate that the superior component 1200 can include a superior keel 1248 that extends from superior bearing surface 1206. During installation, described below, the superior keel 1248 can at least partially engage a keel groove that can be established within a cortical rim of a vertebra. Further, the superior keel 1248 can be coated with a bone-growth promoting substance, e.g., a hydroxyapatite coating formed of calcium phosphate. In a particular embodiment, the superior keel 1248 does not include proteins, e.g., bone morphogenetic protein (BMP). Additionally, the superior keel 1248 can be roughened prior to being coated with the bone-growth promoting substance to further enhance bone on-growth or in-growth. In a particular embodiment, the roughening process can include acid etching; knurling; application of a bead coating (porous or non-porous), e.g., cobalt chrome beads; application of a roughening spray, e.g., titanium plasma spray (TPS); laser blasting; or any other similar process or method.

In a particular embodiment, the superior component 1200, depicted in FIG. 15, can be generally rectangular in shape. For example, the superior component 1200 can have a substantially straight posterior side 1250. A first substantially straight lateral side 1252 and a second substantially straight lateral side 1254 can extend substantially perpendicularly from the posterior side 1250 to an anterior side 1256. In a particular embodiment, the anterior side 1256 can curve outward such that the superior component 1200 is wider through the middle than along the lateral sides 1252, 1254. Further, in a particular embodiment, the lateral sides 1252, 1254 are substantially the same length.

FIG. 15 shows that the superior component 1200 can include a first implant inserter engagement hole 1260 and a second implant inserter engagement hole 1262. In a particular embodiment, the implant inserter engagement holes 1260, 1262 are configured to receive a correspondingly shaped arm that extends from an implant inserter (not shown) that can be used to facilitate the proper installation of an intervertebral prosthetic disc, e.g., the intervertebral prosthetic disc 1100 shown in FIG. 11 through FIG. 16.

In a particular embodiment, the inferior component 1300 includes an inferior support plate 1302 that has an inferior articular surface 1304 and an inferior bearing surface 1306. In a particular embodiment, the inferior articular surface 1304 can be substantially flat and the inferior bearing surface 1306 can be generally curved. In an alternative embodiment, at least a portion of the inferior articular surface 1304 can be generally curved and the inferior bearing surface 1306 can be substantially flat.

In a particular embodiment, after installation, the inferior bearing surface 1306 can be in direct contact with vertebral bone, e.g., cortical bone and cancellous bone. Further, the inferior bearing surface 1306 can be coated with a bone-growth promoting substance, e.g., a hydroxyapatite coating formed of calcium phosphate. In a particular embodiment, the inferior bearing surface 1306 does not include proteins, e.g., bone morphogenetic protein (BMP). Additionally, the inferior bearing surface 1306 can be roughened prior to being coated with the bone-growth promoting substance to further enhance bone on-growth or in-growth. In a particular embodiment, the roughening process can include acid etching; knurling; application of a bead coating (porous or non-porous), e.g., cobalt chrome beads; application of a roughening spray, e.g., titanium plasma spray (TPS); laser blasting; or any other similar process or method.

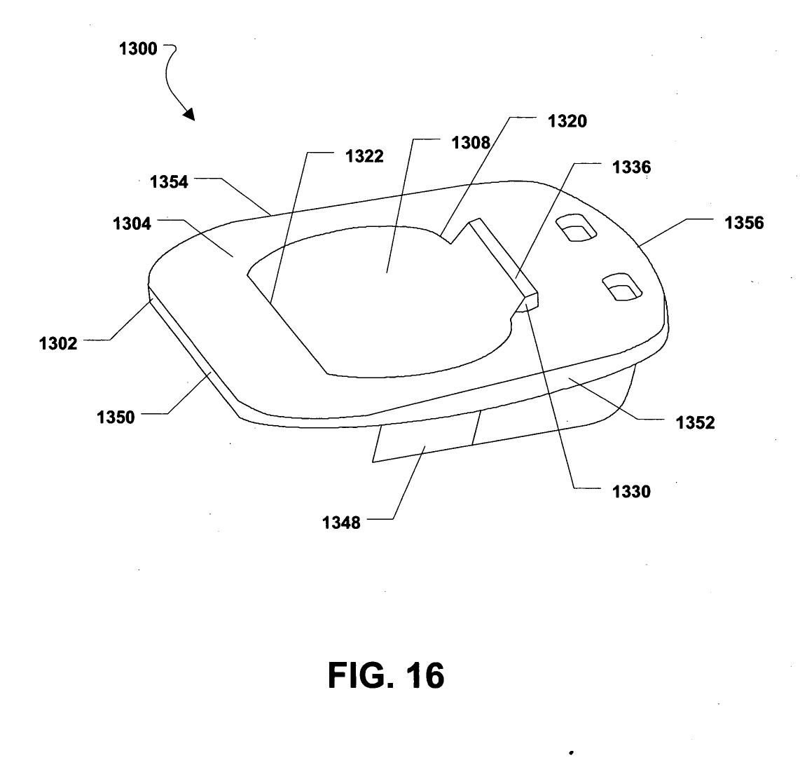

As illustrated in FIG. 16, an inferior depression 1308 is established within the inferior articular surface 1304 of the inferior support plate 1302. In a particular embodiment, the inferior depression 1308 has an arcuate shape. For example, the inferior depression 1308 can have a hemispherical shape, an elliptical shape, a cylindrical shape, or any combination thereof.

As further shown in FIG. 16, the inferior depression 1308 includes an anterior rim 1320 and a poster rim 1322. Further, an inferior nucleus containment rail 1330 extends from the inferior articular surface 1304 adjacent to the anterior rim 1320 of the inferior depression 1308. As shown in FIG. 16, the inferior nucleus containment rail 1330 is an extension of the surface of the inferior depression 1308. In a particular embodiment, the inferior nucleus containment rail 1330 extends into a gap 1334 that can be established between the superior component 1200 and the inferior component 1300 posterior to the nucleus 1400. Further, the inferior nucleus containment rail 1330 can include a slanted upper surface 1336. In a particular embodiment, the slanted upper surface 1336 of the inferior nucleus containment rail 1330 can prevent the inferior nucleus containment rail 1330 from interfering with the motion of the superior component 1200 with respect to the inferior component 1300.

In lieu of, or in addition to, the inferior nucleus containment rail 1330, a superior nucleus containment rail (not shown) can extend from the superior articular surface 1204 of the superior component 1200. In a particular embodiment, the superior nucleus containment rail (not shown) can be configured substantially identical to the inferior nucleus containment rail 1330. In various alternative embodiments (not shown), each or both of the superior component 1200 and the inferior component 1300 can include multiple nucleus containment rails extending from the respective articular surfaces 1204, 1304. The containment rails can be staggered or provided in other configurations based on the perceived need to prevent nucleus migration in a given direction.

FIG. 11 through FIG. 14 and FIG. 16 indicate that the inferior component 1300 can include an inferior keel 1348 that extends from inferior bearing surface 1306. During installation, described below, the inferior keel 1348 can at least partially engage a keel groove that can be established within a cortical rim of a vertebra. Further, the inferior keel 1348 can be coated with a bone-growth promoting substance, e.g., a hydroxyapatite coating formed of calcium phosphate. In a particular embodiment, the inferior keel 1348 does not include proteins, e.g., bone morphogenetic protein (BMP). Additionally, the inferior keel 1348 can be roughened prior to being coated with the bone-growth promoting substance to further enhance bone on-growth or in-growth. In a particular embodiment, the roughening process can include acid etching; knurling; application of a bead coating (porous or non-porous), e.g., cobalt chrome beads; application of a roughening spray, e.g., titanium plasma spray (TPS); laser blasting; or any other similar process or method.

In a particular embodiment, the inferior component 1300, shown in FIG. 16, can be shaped to match the shape of the inferior component 1200, shown in FIG. 15. Further, the inferior component 1300 can be generally rectangular in shape. For example, the inferior component 1300 can have a substantially straight posterior side 1350. A first substantially straight lateral side 1352 and a second substantially straight lateral side 1354 can extend substantially perpendicularly from the posterior side 1350 to an anterior side 1356. In a particular embodiment, the anterior side 1356 can curve outward such that the inferior component 1300 is wider through the middle than along the lateral sides 1352, 1354. Further, in a particular embodiment, the lateral sides 1352, 1354 are substantially the same length.

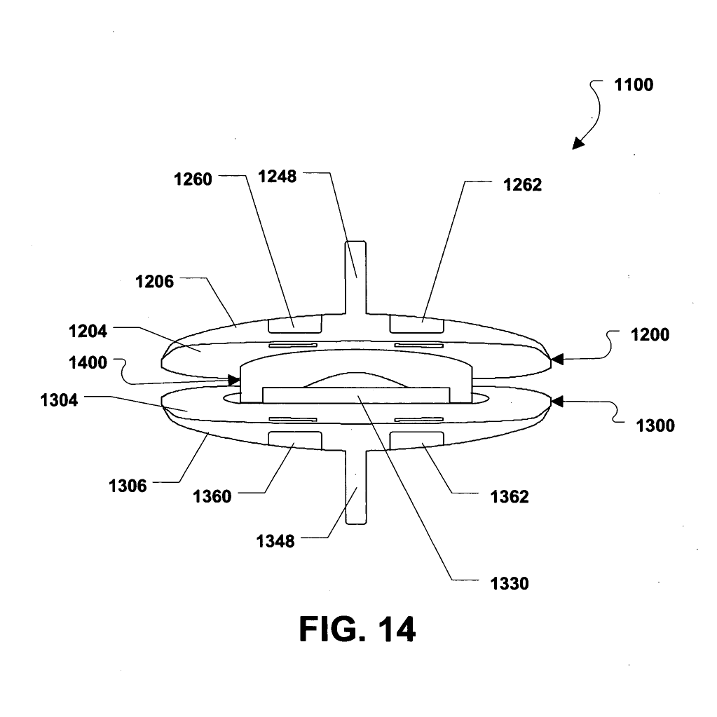

FIG. 14 shows that the inferior component 1300 can include a first implant inserter engagement hole 1360 and a second implant inserter engagement hole 1362. In a particular embodiment, the implant inserter engagement holes 1360, 1362 are configured to receive a correspondingly shaped arm that extends from an implant inserter (not shown) that can be used to facilitate the proper installation of an intervertebral prosthetic disc, e.g., the intervertebral prosthetic disc 1100 shown in FIG. 11 through FIG. 16.

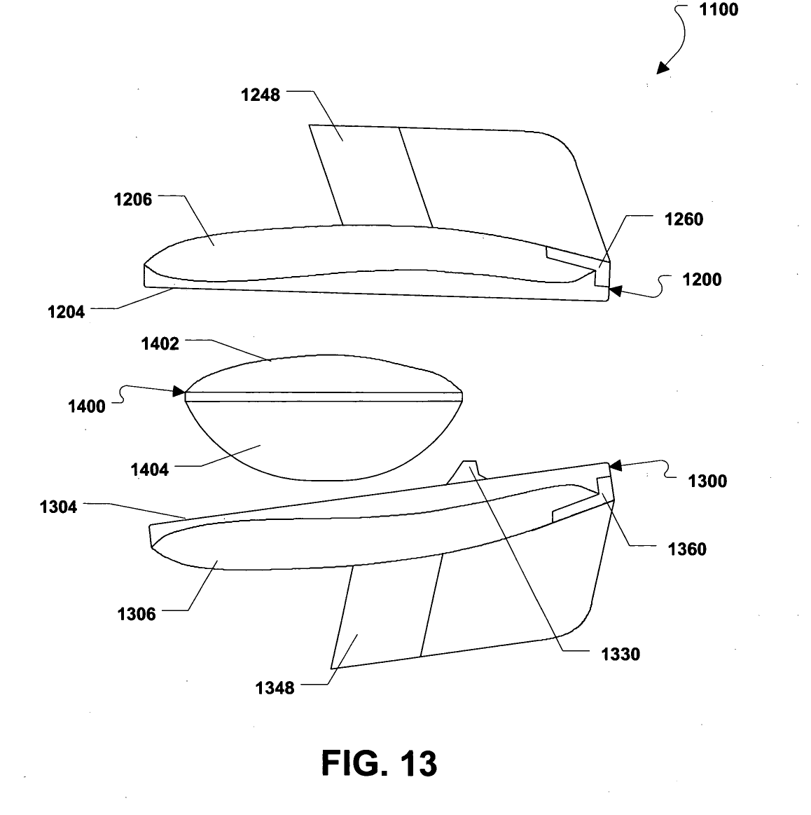

FIG. 13 shows that the nucleus 1400 can include a superior bearing surface 1402 and an inferior bearing surface 1404. In a particular embodiment, the superior bearing surface 1402 and the inferior bearing surface 1404 can each have an arcuate shape. For example, the superior bearing surface 1402 of the nucleus 1400 and the inferior bearing surface 1404 of the nucleus 1400 can have a hemispherical shape, an elliptical shape, a cylindrical shape, or any combination thereof. Further, in a particular embodiment, the superior bearing surface 1402 can be curved to match the superior depression 1208 of the superior component 1200. Also, in a particular embodiment, the inferior bearing surface 1404 of the nucleus can be curved to match the inferior depression 1308 of the inferior component 1300.

As shown in FIG. 11, the superior bearing surface 1402 of the nucleus 1400 can engage the superior depression 1208 and allow the superior component 1200 to move relative to the nucleus 1400. Also, the inferior bearing surface 1404 of the nucleus 1400 can engage the inferior depression 1308 and allow the inferior component 1300 to move relative to the nucleus 1400. Accordingly, the nucleus 1400 can engage the superior component 1200 and the inferior component 1300 and the nucleus 1400 can allow the superior component 1200 to rotate with respect to the inferior component 1300.

In a particular embodiment, the inferior nucleus containment rail 1330 on the inferior component 1300 can prevent the nucleus 1400 from migrating, or moving, with respect to the superior component 1200, the inferior component 1300, or a combination thereof. In other words, the inferior nucleus containment rail 1330 can prevent the nucleus 1400 from moving out of the superior depression 1208, the inferior depression 1308, or a combination thereof.

Further, the inferior nucleus containment rail 1330 can prevent the nucleus 1400 from being expelled from the intervertebral prosthetic device 1100. In other words, the inferior nucleus containment rail 1330 on the inferior component 1300 can prevent the nucleus 1400 from being completely ejected from the intervertebral prosthetic device 1100 while the superior component 1200 and the inferior component 1300 move with respect to each other.

In a particular embodiment, the overall height of the intervertebral prosthetic device 1100 can be in a range from fourteen millimeters to forty-six millimeters (14-46 mm). Further, the installed height of the intervertebral prosthetic device 1100 can be in a range from eight millimeters to sixteen millimeters (8-16 mm). In a particular embodiment, the installed height can be substantially equivalent to the distance between an inferior vertebra and a superior vertebra when the intervertebral prosthetic device 1100 is installed there between.

In a particular embodiment, the length of the intervertebral prosthetic device 1100, e.g., along a longitudinal axis, can be in a range from thirty millimeters to forty millimeters (30-40 mm). Additionally, the width of the intervertebral prosthetic device 1100, e.g., along a lateral axis, can be in a range from twenty-five millimeters to forty millimeters (25-40 mm). Moreover, in a particular embodiment, each keel 1248, 1348 can have a height in a range from three millimeters to fifteen millimeters (3-15 mm).

### Description of a Third Embodiment

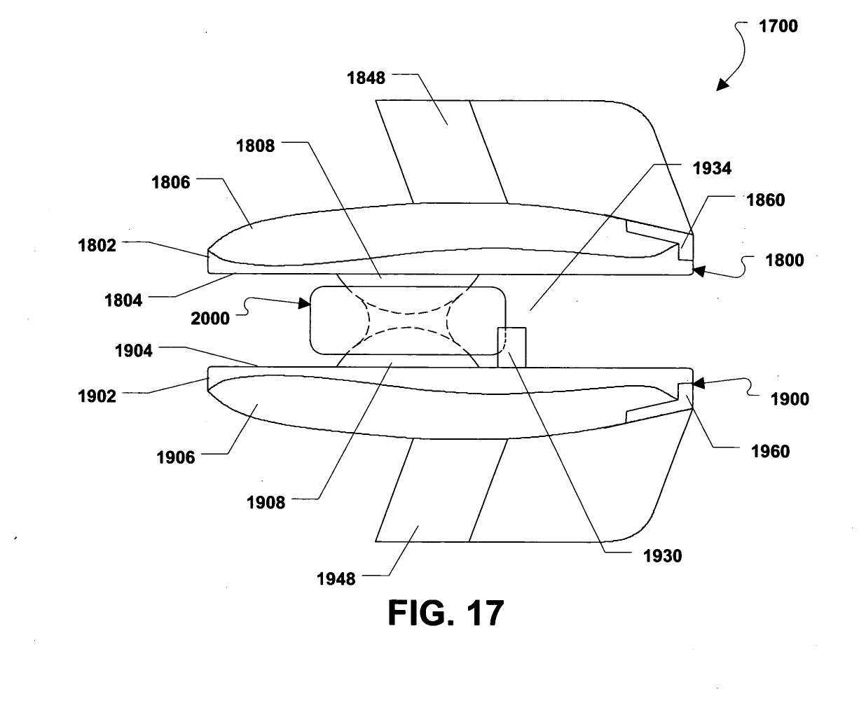

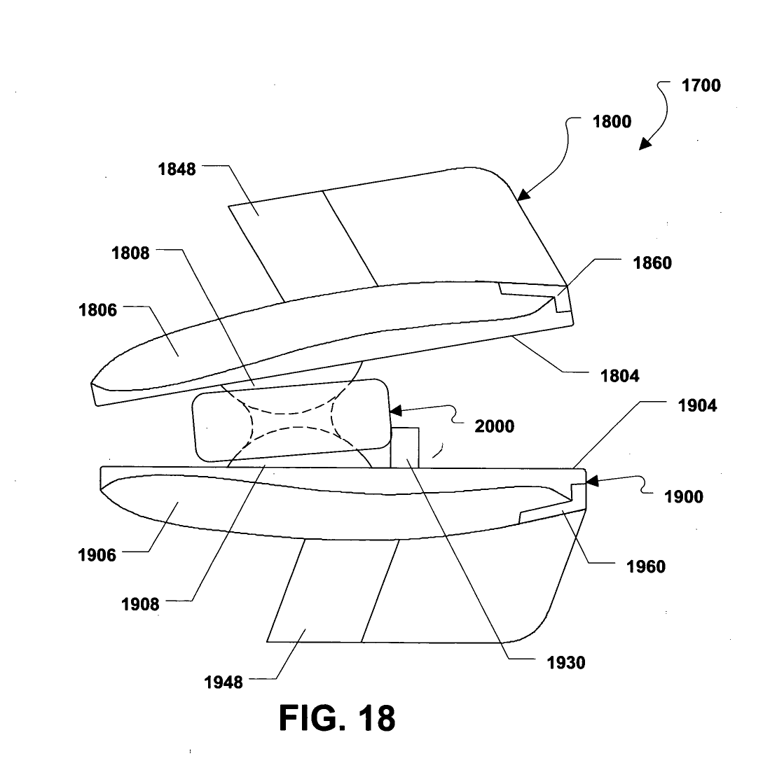

Referring to FIGS. 17 through 22, a third embodiment of an intervertebral prosthetic disc is shown and is generally designated 1700. As illustrated, the intervertebral prosthetic disc 1700 includes a superior component 1800, an inferior component 1900, and a nucleus 2000 disposed, or otherwise installed, there between. In a particular embodiment, the articular halves 1800, 1900 and the nucleus 2000 can be made from one or more extended use approved medical materials. For example, the materials can be metal containing materials, polymer materials, or composite materials that include metals, polymers, or combinations of metals and polymers.

In a particular embodiment, the metal containing materials can be metals. Further, the metal containing materials can be ceramics. Also, the metals can be pure metals or metal alloys. The pure metals can include titanium. Moreover, the metal alloys can include stainless steel, a cobalt-chrome-molybdenum alloy, e.g., ASTM F-999 or ASTM F-75, a titanium alloy, or a combination thereof.

The polymer materials can include polyurethane materials, polyolefin materials, polyether materials, silicone materials, or a combination thereof. Further, the polyolefin materials can include polypropylene, polyethylene, halogenated polyolefin, flouropolyolefin, or a combination thereof. The polyether materials can include polyetherketone (PEK), polyetheretherketone (PEEK), polyetherketoneketone (PEKK), polyaryletherketone (PAEK), or a combination thereof. Alternatively, the articular halves 1800, 1900 can be made from any other substantially rigid biocompatible materials.

In a particular embodiment, the superior component 1800 includes a superior support plate 1802 that has a superior articular surface 1804 and a superior bearing surface 1806. In a particular embodiment, the superior articular surface 1804 can be substantially flat and the superior bearing surface 1806 can be generally curved. In an alternative embodiment, at least a portion of the superior articular surface 1804 can be generally curved and the superior bearing surface 1806 can be substantially flat.

In a particular embodiment, after installation, the superior bearing surface 1806 can be in direct contact with vertebral bone, e.g., cortical bone and cancellous bone. Further, the superior bearing surface 1806 can be coated with a bone-growth promoting substance, e.g., a hydroxyapatite coating formed of calcium phosphate. In a particular embodiment, the superior bearing surface 1806 does not include proteins, e.g., bone morphogenetic protein (BMP). Additionally, the superior bearing surface 1806 can be roughened prior to being coated with the bone-growth promoting substance to further enhance bone on-growth or in-growth. In a particular embodiment, the roughening process can include acid etching; knurling; application of a bead coating (porous or non-porous), e.g., cobalt chrome beads; application of a roughening spray, e.g., titanium plasma spray (TPS); laser blasting; or any other similar process or method.

As illustrated in FIG. 17 through FIG. 21, a superior projection 1808 extends from the superior articular surface 1804 of the superior support plate 1802. In a particular embodiment, the superior projection 1808 has an arcuate shape. For example, the superior depression 1808 can have a hemispherical shape, an elliptical shape, a cylindrical shape, or any combination thereof.

FIG. 17 through FIG. 21 indicate that the superior component 1800 can include a superior keel 1848 that extends from superior bearing surface 1806. During installation, described below, the superior keel 1848 can at least partially engage a keel groove that can be established within a cortical rim of a vertebra. Further, the superior keel 1848 can be coated with a bone-growth promoting substance, e.g., a hydroxyapatite coating formed of calcium phosphate. In a particular embodiment, the superior keel 1848 does not include proteins, e.g., bone morphogenetic protein (BMP). Additionally, the superior keel 1848 can be roughened prior to being coated with the bone-growth promoting substance to further enhance bone on-growth or in-growth. In a particular embodiment, the roughening process can include acid etching; knurling; application of a bead coating (porous or non-porous), e.g., cobalt chrome beads; application of a roughening spray, e.g., titanium plasma spray (TPS); laser blasting; or any other similar process or method.

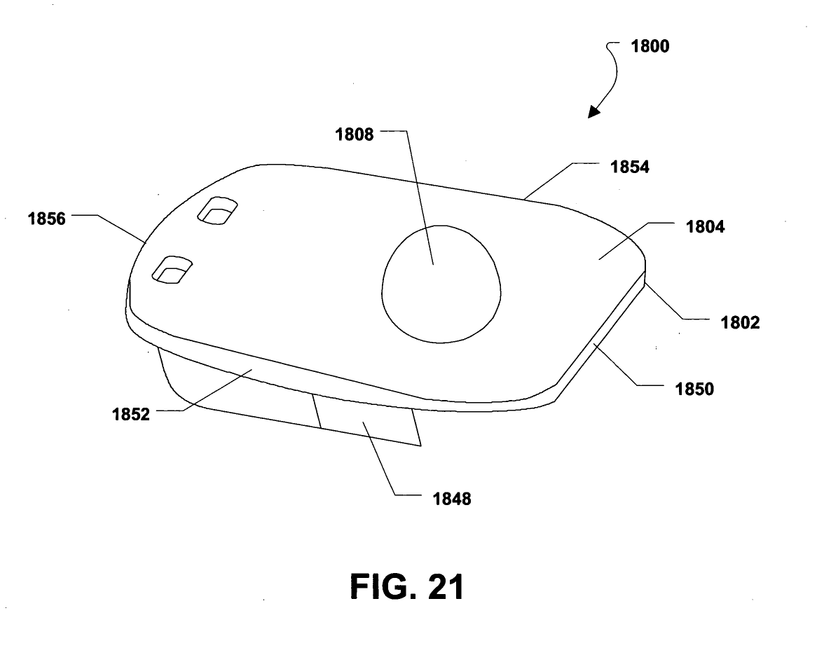

In a particular embodiment, the superior component 1800, depicted in FIG. 21, can be generally rectangular in shape. For example, the superior component 1800 can have a substantially straight posterior side 1850. A first substantially straight lateral side 1852 and a second substantially straight lateral side 1854 can extend substantially perpendicularly from the posterior side 1850 to an anterior side 1856. In a particular embodiment, the anterior side 1856 can curve outward such that the superior component 1800 is wider through the middle than along the lateral sides 1852, 1854. Further, in a particular embodiment, the lateral sides 1852, 1854 are substantially the same length.

FIG. 21 shows that the superior component 1800 can include a first implant inserter engagement hole 1860 and a second implant inserter engagement hole 1862. In a particular embodiment, the implant inserter engagement holes 1860, 1862 are configured to receive a correspondingly shaped arm that extends from an implant inserter (not shown) that can be used to facilitate the proper installation of an intervertebral prosthetic disc, e.g., the intervertebral prosthetic disc 1700 shown in FIG. 17 through FIG. 22.

In a particular embodiment, the inferior component 1900 includes an inferior support plate 1902 that has an inferior articular surface 1904 and an inferior bearing surface 1906. In a particular embodiment, the inferior articular surface 1904 can be substantially flat and the inferior bearing surface 1906 can be generally curved. In an alternative embodiment, at least a portion of the inferior articular surface 1904 can be generally curved and the inferior bearing surface 1906 can be substantially flat.

In a particular embodiment, after installation, the inferior bearing surface 1906 can be in direct contact with vertebral bone, e.g., cortical bone and cancellous bone. Further, the inferior bearing surface 1906 can be coated with a bone-growth promoting substance, e.g., a hydroxyapatite coating formed of calcium phosphate. In a particular embodiment, the inferior bearing surface 1906 does not include proteins, e.g., bone morphogenetic protein (BMP). Additionally, the inferior bearing surface 1906 can be roughened prior to being coated with the bone-growth promoting substance to further enhance bone on-growth or in-growth. In a particular embodiment, the roughening process can include acid etching; knurling; application of a bead coating (porous or non-porous), e.g., cobalt chrome beads; application of a roughening spray, e.g., titanium plasma spray (TPS); laser blasting; or any other similar process or method.

As illustrated in FIG. 16, an inferior projection 1908 can extend from the inferior articular surface 1904 of the inferior support plate 1902. In a particular embodiment, the inferior projection 1908 has an arcuate shape. For example, the inferior projection 1908 can have a hemispherical shape, an elliptical shape, a cylindrical shape, or any combination thereof.

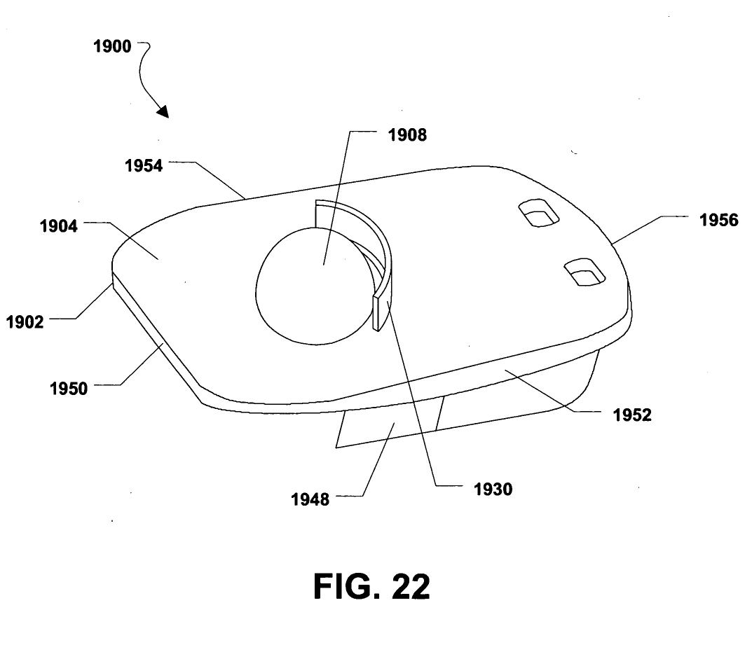

As further shown, an inferior nucleus containment rail 1930 can extend from the inferior articular surface 1904 adjacent to the inferior projection 1908. As shown in FIG. 22, the inferior nucleus containment rail 1930 is a curved wall that extends from the inferior articular surface 1904. In a particular embodiment, the inferior nucleus containment rail 1930 can be curved to match the shape, or curvature, of the inferior projection 1908. Alternatively, the inferior nucleus containment rail 1930 can be curved to match the shape, or curvature, of the nucleus 2000. In a particular embodiment, the inferior nucleus containment rail 1930 extends into a gap 1934 that can be established between the superior component 1800 and the inferior component 1900 posterior to the nucleus 2000.

In lieu of, or in addition to, the inferior nucleus containment rail 1930, a superior nucleus containment rail (not shown) can extend from the superior articular surface 1804 of the superior component 1800. In a particular embodiment, the superior nucleus containment rail (not shown) can be configured substantially identical to the inferior nucleus containment rail 1930. In various alternative embodiments (not shown), each or both of the superior component 1800 and the inferior component 1900 can include multiple nucleus containment rails extending from the respective articular surfaces 1804, 1904. The containment rails can be staggered or provided in other configurations based on the perceived need to prevent nucleus migration in a given direction.

FIG. 17 through FIG. 20 and FIG. 22 indicate that the inferior component 1900 can include an inferior keel 1948 that extends from inferior bearing surface 1906. During installation, described below, the inferior keel 1948 can at least partially engage a keel groove that can be established within a cortical rim of a vertebra. Further, the inferior keel 1948 can be coated with a bone-growth promoting substance, e.g., a hydroxyapatite coating formed of calcium phosphate. In a particular embodiment, the inferior keel 1948 does not include proteins, e.g., bone morphogenetic protein (BMP). Additionally, the inferior keel 1948 can be roughened prior to being coated with the bone-growth promoting substance to further enhance bone on-growth or in-growth. In a particular embodiment, the roughening process can include acid etching; knurling; application of a bead coating (porous or non-porous), e.g., cobalt chrome beads; application of a roughening spray, e.g., titanium plasma spray (TPS); laser blasting; or any other similar process or method.

In a particular embodiment, the inferior component 1900, shown in FIG. 22, can be shaped to match the shape of the inferior component 1800, shown in FIG. 21. Further, the inferior component 1900 can be generally rectangular in shape. For example, the inferior component 1900 can have a substantially straight posterior side 1950. A first substantially straight lateral side 1952 and a second substantially straight lateral side 1954 can extend substantially perpendicularly from the posterior side 1950 to an anterior side 1956. In a particular embodiment, the anterior side 1956 can curve outward such that the inferior component 1900 is wider through the middle than along the lateral sides 1952, 1954. Further, in a particular embodiment, the lateral sides 1952, 1954 are substantially the same length.

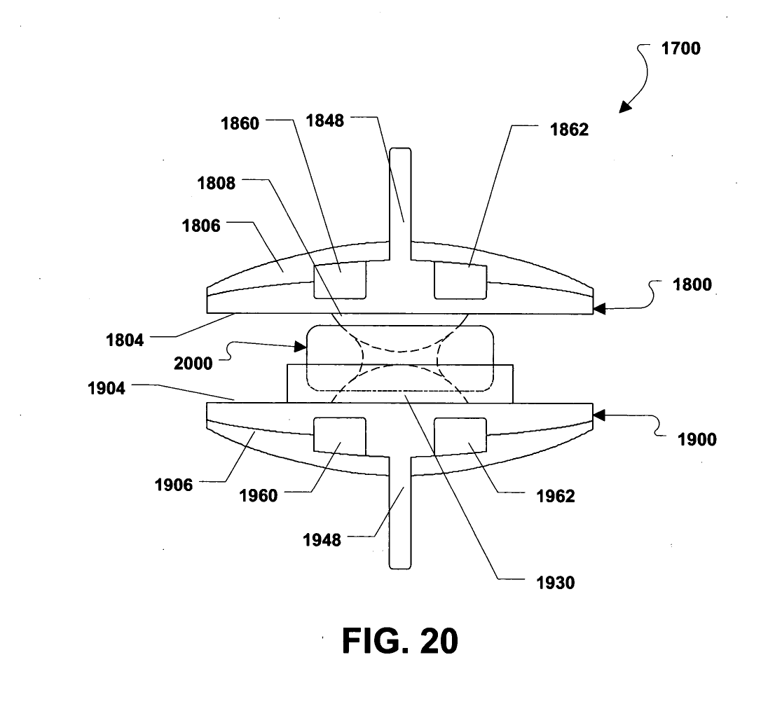

FIG. 20 and FIG. 22 show that the inferior component 1900 can include a first implant inserter engagement hole 1960 and a second implant inserter engagement hole 1962. In a particular embodiment, the implant inserter engagement holes 1960, 1962 are configured to receive a correspondingly shaped arm that extends from an implant inserter (not shown) that can be used to facilitate the proper installation of an intervertebral prosthetic disc, e.g., the intervertebral prosthetic disc 1700 shown in FIG. 17 through FIG. 22.

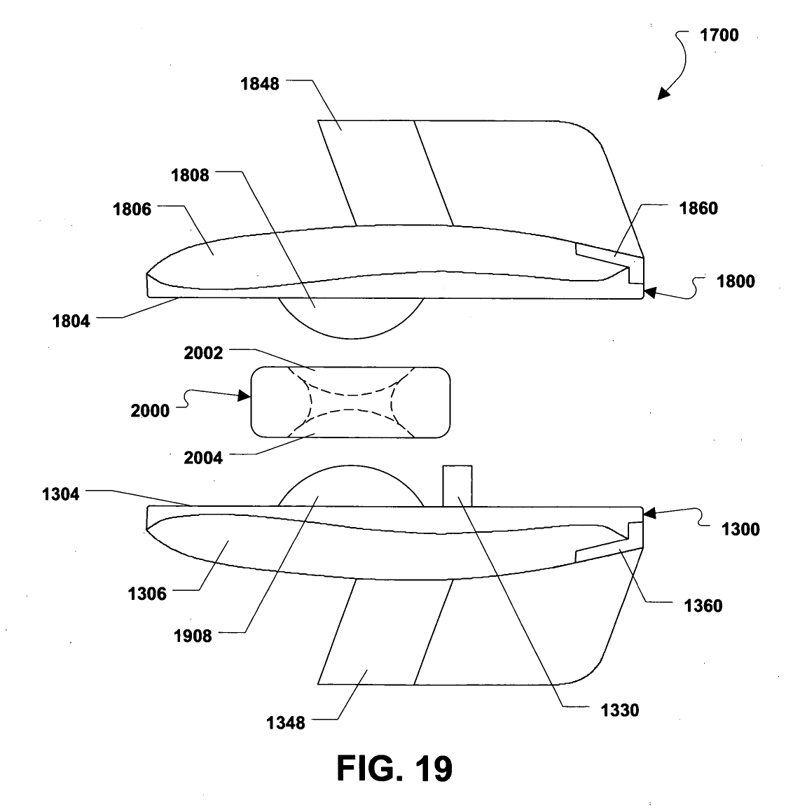

FIG. 19 shows that the nucleus 2000 can include a superior depression 2002 and an inferior depression 2004. In a particular embodiment, the superior depression 2002 and the inferior depression 2004 can each have an arcuate shape. For example, the superior depression 2002 of the nucleus 2000 and the inferior depression 2004 of the nucleus 2000 can have a hemispherical shape, an elliptical shape, a cylindrical shape, or any combination thereof. Further, in a particular embodiment, the superior depression 2002 can be curved to match the superior projection 1808 of the superior component 1800. Also, in a particular embodiment, the inferior depression 2004 of the nucleus 2000 can be curved to match the inferior projection 1908 of the inferior component 1900.

As shown in FIG. 17, the superior depression 2002 of the nucleus 2000 can engage the superior projection 1808 and allow the superior component 1800 to move relative to the nucleus 2000. Also, the inferior depression 2004 of the nucleus 2000 can engage the inferior projection 1908 and allow the inferior component 1900 to move relative to the nucleus 2000. Accordingly, the nucleus 2000 can engage the superior component 1800 and the inferior component 1900, and the nucleus 2000 can allow the superior component 1800 to rotate with respect to the inferior component 1900.

In a particular embodiment, the inferior nucleus containment rail 1930 on the inferior component 1900 can prevent the nucleus 2000 from migrating, or moving, with respect to the superior component 1800 and the inferior component 1900. In other words, the inferior nucleus containment rail 1930 can prevent the nucleus 2000 from moving off of the superior projection 1808, the inferior projection 1908, or a combination thereof.

Further, the inferior nucleus containment rail 1930 can prevent the nucleus 2000 from being expelled from the intervertebral prosthetic device 1700. In other words, the inferior nucleus containment rail 1930 on the inferior component 1900 can prevent the nucleus 2000 from being completely ejected from the intervertebral prosthetic device 1700 while the superior component 1800 and the inferior component 1900 move with respect to each other.

In a particular embodiment, the overall height of the intervertebral prosthetic device 1700 can be in a range from fourteen millimeters to forty-six millimeters (14-46 mm). Further, the installed height of the intervertebral prosthetic device 1700 can be in a range from eight millimeters to sixteen millimeters (8-16 mm). In a particular embodiment, the installed height can be substantially equivalent to the distance between an inferior vertebra and a superior vertebra when the intervertebral prosthetic device 1700 is installed there between.

In a particular embodiment, the length of the intervertebral prosthetic device 1700, e.g., along a longitudinal axis, can be in a range from thirty millimeters to forty millimeters (30-40 mm). Additionally, the width of the intervertebral prosthetic device 1700, e.g., along a lateral axis, can be in a range from twenty-five millimeters to forty millimeters (25-40 mm). Moreover, in a particular embodiment, each keel 1848, 1948 can have a height in a range from three millimeters to fifteen millimeters (3-15 mm).

### CONCLUSION

With the configuration of structure described above, the intervertebral prosthetic disc according to one or more of the embodiments provides a device that may be implanted to replace a natural intervertebral disc that is diseased, degenerated, or otherwise damaged. The intervertebral prosthetic disc can be disposed within an intervertebral space between an inferior vertebra and a superior vertebra. Further, after a patient fully recovers from a surgery to implant the intervertebral prosthetic disc, the intervertebral prosthetic disc can provide relative motion between the inferior vertebra and the superior vertebra that closely replicates the motion provided by a natural intervertebral disc. Accordingly, the intervertebral prosthetic disc provides an alternative to a fusion device that can be implanted within the intervertebral space between the inferior vertebra and the superior vertebra to fuse the inferior vertebra and the superior vertebra and prevent relative motion there between.

The intervertebral prosthetic disc according to one or more of the embodiments, disclosed herein, includes at least one nucleus containment feature, e.g., one or more superior or inferior nucleus containment posts or one or more superior or inferior nucleus containment rails. The nucleus containment feature can prevent nucleus migration, nucleus expulsion, or any other unwanted movement of the nucleus with respect to the superior component and the inferior component while the superior component and the inferior component move relative to each other. Additionally, other multi-level intervertebral prosthetic disc can include similar containment structures to prevent nucleus migration, nucleus expulsion, or any other unwanted nucleus movement.

The above-disclosed subject matter is to be considered illustrative, and not restrictive, and the appended claims are intended to cover all such modifications, enhancements, and other embodiments that fall within the true spirit and scope of the present invention. For example, it is noted that the articular halves in the exemplary embodiments described herein are referred to as “superior” and “inferior” for illustrative purposes only and that one or more of the features described as part of or attached to a respective component may be provided as part of or attached to the other component in addition or in the alternative. Thus, to the maximum extent allowed by law, the scope of the present invention is to be determined by the broadest permissible interpretation of the following claims and their equivalents, and shall not be restricted or limited by the foregoing detailed description.

1. An intervertebral prosthetic disc to be installed within an intervertebral space between an inferior vertebra and a superior vertebra, the intervertebral prosthetic disc comprising:
a superior component configured to engage the superior vertebra;
an inferior component configured to engage the inferior vertebra;
a nucleus disposed between the superior component and the inferior component, wherein the nucleus is configured to allow relative motion between the superior component and the inferior component; and
a nucleus containment feature configured to prevent the nucleus from migrating with respect to the superior component and the inferior component without interfering with the relative motion between the superior component and the inferior component in any direction.

2. The intervertebral prosthetic disc of claim 1, wherein the nucleus containment feature is configured to prevent the nucleus from being expelled from between the superior component and the inferior component.

3. The intervertebral prosthetic disc of claim 2, wherein the superior component comprises:
a superior support plate having a superior bearing surface and a superior articular surface; and
a superior depression established within the superior articular surface, wherein the superior depression is configured to receive a superior bearing surface of the nucleus and wherein the superior depression includes an anterior rim and a posterior rim.

4. The intervertebral prosthetic disc of claim 3, wherein the nucleus containment feature comprises a superior nucleus containment post that extends from the superior articular surface near the anterior rim of the superior depression.

5. The intervertebral prosthetic disc of claim 4, wherein the superior nucleus containment post includes a slanted upper surface.

6. The intervertebral prosthetic disc of claim 3, wherein the nucleus containment feature comprises a superior nucleus containment rail that extends from the superior articular surface along the anterior rim of the superior depression.

7. The intervertebral prosthetic disc of claim 6, wherein the superior nucleus containment rail includes a slanted upper surface.

8. The intervertebral prosthetic disc of claim 6, wherein the at least a portion of the superior nucleus containment rail is a continuation of an inner surface of the superior depression.

9. The intervertebral prosthetic disc of claim 2, wherein the inferior component comprises:
an inferior support plate having an inferior bearing surface and a inferior articular surface; and
an inferior depression established within the inferior articular surface, wherein the inferior depression is configured to receive an inferior bearing surface of the nucleus and wherein the inferior depression includes an anterior rim and a posterior rim.

10. The intervertebral prosthetic disc of claim 9, wherein the nucleus containment feature further comprises an inferior nucleus containment post that extends from the inferior articular surface near the anterior rim of the inferior depression.

11. The intervertebral prosthetic disc of claim 10, wherein the inferior nucleus containment post includes a slanted upper surface.

12. The intervertebral prosthetic disc of claim 9, wherein the nucleus containment feature comprises an inferior nucleus containment rail that extends from the inferior articular surface along the anterior rim of the inferior depression.

13. The intervertebral prosthetic disc of claim 12, wherein the inferior nucleus containment rail includes a slanted upper surface.

14. The intervertebral prosthetic disc of claim 12, wherein the at least a portion of the inferior nucleus containment rail is a continuation of an inner surface of the inferior depression.

15. An intervertebral prosthetic disc to be installed within an intervertebral space between an inferior vertebra and a superior vertebra, the intervertebral prosthetic disc comprising:
a superior component configured to engage the superior vertebra, the superior component including a superior depression established therein, the superior depression including an anterior rim and a posterior rim;
an inferior component configured to engage the inferior vertebra, the inferior component including an inferior depression established therein, the inferior depression including an anterior rim and a posterior rim;
a nucleus disposed between the superior component and the inferior component, wherein the nucleus is configured to engage the superior depression and the inferior depression and wherein the nucleus is configured to allow relative motion between the superior component and the inferior component; and
a superior nucleus containment post extending from the superior component, wherein the superior nucleus containment post is configured to prevent the nucleus from migrating with respect to the superior component and the inferior component.

16.-19. (canceled)

20. The intervertebral prosthetic disc of claim 15, further comprising an inferior nucleus containment post extending from the inferior component, wherein the inferior nucleus containment post is configured to prevent the nucleus from migrating with respect to the superior component and the inferior component.

21.-23. (canceled)

24. An intervertebral prosthetic disc to be installed within an intervertebral space between adjacent first and second vertebrae, the intervertebral prosthetic disc comprising:
a first component configured to engage the first vertebra, the first component including a depression established therein, the depression including an anterior rim and a posterior rim;
a second component configured to engage the second vertebra, the second component including a depression established therein, the depression including an anterior rim and a posterior rim;
a nucleus disposed between the first component and the second component, wherein the nucleus is configured to engage the depression of the first component and the depression of the second component and wherein the nucleus is configured to allow relative motion between the first component and the second component; and
a first nucleus containment rail extending from at least one of the first component or the second component, wherein the first nucleus containment rail is configured to prevent the nucleus from migrating with respect to the first component and the second component.

25.-28. (canceled)

29. The intervertebral prosthetic disc of claim 24, further comprising a second nucleus containment rail that extends from the second component along the anterior rim of the depression of the second component.

30.-31. (canceled)

32. An intervertebral prosthetic disc to be installed within an intervertebral space between an inferior vertebra and a superior vertebra, the intervertebral prosthetic disc comprising:
a superior component configured to engage the superior vertebra, the superior component including a superior projection extending therefrom;
an inferior component configured to engage the inferior vertebra, the inferior component including an inferior projection extending therefrom;
a nucleus disposed between the superior component and the inferior component, wherein the nucleus is configured to engage the superior projection and the inferior projection and wherein the nucleus is configured to allow relative motion between the superior component and the inferior component; and
an inferior nucleus containment rail extending from the inferior component, wherein the inferior nucleus containment rail is configured to prevent the nucleus from migrating with respect to the superior component and the inferior component.

33. The intervertebral prosthetic disc of claim 32, wherein the inferior nucleus containment rail is configured to prevent the nucleus from being expelled from the intervertebral prosthetic disc.

34. The intervertebral prosthetic disc of claim 33, wherein the inferior nucleus containment rail extends from the inferior component adjacent to the inferior projection.

35. The intervertebral prosthetic disc of claim 34, wherein the inferior nucleus containment rail is curved.

36. The intervertebral prosthetic disc of claim 35, wherein the inferior nucleus containment rail is curved to match a curvature of the inferior projection.

37. The intervertebral prosthetic disc of claim 35, wherein the inferior nucleus containment rail is curved to match a curvature of the nucleus.

38.-43. (canceled)
